(19) United States
(12) Patent Application Publication
(10) Pub. No.: US 2026/0315248 A1
(43) Pub. Date: Oct. 15, 2026

(54) SWITCHING VALVE

(71) Applicant: OTTOBOCK SE & CO. KGAA, Duderstadt (DE)

(72) Inventors: Andreas SCHUH, Duderstadt (DE); Thorsten GROHS, Duderstadt (DE); Martin PUSCH, Duderstadt (DE); Sven KALTENBORN, Duderstadt (DE)

(21) Appl. No.: 19/483,401

(22) PCT Filed: May 7, 2024
(86) PCT No.: PCT/EP2024/062605

§ 371 Date: Nov. 11, 2025

(30) Foreign Application Priority Data
May 12, 2023 (DE) 10 2023 112 610.5
Oct. 5, 2023 (DE) 10 2023 127 105.9

(51) Int. Cl. A61F 2/66; A61F 2/74

(52) U.S. Cl. CPC A61F 2/66; A61F 2/748; A61F 2002/6614

(57) ABSTRACT

The invention relates to a prosthetic foot with a foot part, a lower leg part (36) arranged pivotably on the foot part (38) and a hydraulic system, wherein the hydraulic system has two hydraulic chambers (40) that are fluidically connected by a hydraulic line, wherein a switching valve is located in the hydraulic line and has a valve housing (2) with an inlet connection (6) and an outlet connection (8), and has a switching shaft (4) which can be moved in a shaft chamber of the valve housing (2) and which can be brought into a first position in which the inlet connection (6) is fluidically connected to the outlet connection (8) and into a second position in which the inlet connection (6) is fluidically separated from the outlet connection (8), wherein an actuating element (16) is arranged at least at one end of the switching shaft (4), wherein a seal (20) is arranged between the actuating element (16) and the valve housing (2) and seals an intermediate space between the actuating element (16) and the valve housing (2) in both positions of the switching shaft (4).

The invention relates to a prosthetic foot with a foot part, a lower leg part pivotably arranged on the foot part, and a hydraulic system, the hydraulic system comprising two hydraulic chambers that are fluidically connected by a hydraulic line, wherein a switch valve is located in the hydraulic line that comprises a valve housing with an inlet connection and an outlet connection and a switch shaft that can be moved in a shaft chamber of the valve housing, wherein said switch shaft can be brought into a first position, in which the inlet connection is fluidically connected to the outlet connection, and into a second position, in which the inlet connection is fluidically separated from the outlet connection.

In many prosthetic feet, hydraulic systems are often used to damp the movement of the foot part relative to the lower leg part and/or to transfer forces from an actuator, thus inducing movements of the components relative to each other. In these hydraulic systems, valves are needed and used for various reasons. The hydraulic system comprises two hydraulic chambers, one of which can be referred to as an extension chamber and the other as a flexion chamber. In one preferred embodiment, both hydraulic chambers are arranged in a single cylinder and separated from each other by a moveable piston. The piston and the cylinder are coordinated in such a way and correspondingly designed in shape and contour such that the piston can be displaced in the cylinder. This movement of the piston can be a movement in a straight line. Preferably, the movement of the piston in the cylinder is a swivel movement, preferably carried out about the swivel axis, to enable the foot part and the lower leg part to be swivelled relative to one another. If the foot part is moved relative to the lower leg part, the piston inside the cylinder is also moved. This increases the volume of one hydraulic chamber and reduces the volume of the other hydraulic chamber. Hydraulic fluid of the hydraulic system is therefore guided out of the hydraulic chamber reducing in size into the hydraulic chamber increasing in size. In the process, the hydraulic fluid has to be guided through the switch valve.

The type of valve required for such hydraulic systems is a switch valve. Switch valves are used in a hydraulic line and are configured to open or close this hydraulic line. Switch valves according to the preamble therefore feature a movable switch shaft, which can be brought into a first position and into a second position relative to the valve housing. In the first position, the inlet connection of the valve housing is fluidically connected to the outlet connection of the valve housing. This means that hydraulic liquid can be moved through the switch valve. It enters the valve housing through the inlet connection and leaves the valve housing through the outlet connection. This is preferably possible in both directions such that it is not clear from the switch valve alone which of the two connections is used as an inlet connection and which is used as an outlet connection.

In the second position, the inlet connection is fluidically separated from the outlet connection. This means that it is not possible for the hydraulic fluid to be moved through the switch valve. This allows the hydraulic line to be completely blocked, which is advantageous and desired in many applications in the field of orthopedic technology.

The application of a hydraulic system according to the invention and of a switch valve according to the preamble in a hydraulic line of this hydraulic system is a prosthetic foot that has a foot part and an ankle part. Both are joined together via an artificial joint, a prosthetic ankle joint. If the foot part is swivelled about the swivel axis of this joint, different heel heights of different shoes can be taken into account. However, this movement between foot part and ankle part, which is required if the heel height is to be adjusted, is not always desired and advantageous. Once the heel height has been set, a further swivelling should be rendered impossible. This can be achieved by a switch valve of the type described here. If the switch shaft is brought into the first position relative to the valve housing, the foot part can be swivelled relative to the ankle part. In the process, hydraulic fluid is usually pushed from one hydraulic chamber of the hydraulic system into another hydraulic chamber of the hydraulic system. To this end, the hydraulic fluid has to flow through the hydraulic line in which the switch valve of the type described here is arranged. When the switch shaft is in the first position, the hydraulic fluid can flow between the chambers and the heel height of the prosthetic foot can be adjusted.

If, however, the switch shaft is brought into the second position, the flow through the hydraulic line is suppressed, as described above. The inlet connection of the valve housing is fluidically separated from the outlet connection of the valve housing. As a result, no hydraulic fluid can be guided from one hydraulic chamber of the hydraulic system into the other hydraulic chamber of the hydraulic system, meaning that the foot part of the prosthetic foot cannot be swivelled relative to the ankle part either. Such a system and a corresponding switch valve according to the preamble is known, for example, from U.S. Pat. No. 10821,007B2 .

In this embodiment, the switch shaft is arranged such that it can be displaced along its longitudinal direction, wherein the first position and the second position can be held by a so-called “ball-lock” solution. The switch shaft is held in a mount containing two circumferential grooves, each of which contains three balls that are spring-loaded radially inwards. If the switch shaft is moved, they are pushed outwards by a further ball until the switch shaft has reached the respective position. In this respective position, the balls are pushed radially inwards due to the force exerted by the springs, thus achieving a bistable state. The switch shaft has a first sealing element, particularly a seal, particularly preferably a ring seal, which is arranged on the switch shaft in such a way that in the second position of the switch shaft, it rests against the inner wall of a shaft chamber in which the switch shaft is arranged, thus rendering a flow of hydraulic fluid from the inlet connection to the outlet connection impossible. The switch shaft also has two further seals, preferably two further ring seals, which outwardly seal the shaft chamber where the hydraulic fluid is located and the prevailing pressure in the hydraulic system prevails.

The disadvantage is that such a system and such a switch valve are vulnerable to contamination from outside and, in particular, are not suited to contact with water, especially salt water. This makes it virtually impossible, for example, for the wearer of an orthopedic device in which such a switch valve is used to bathe with the device, for example to swim in the sea.

The invention is therefore based on the task of proposing a switch valve that eliminates or at least reduces the disadvantages of the prior art.

The invention solves the task addressed by way of a prosthetic foot according to the preamble of claim 1, which is characterized in that an actuation element is arranged at at least one end of the switch shaft, wherein a seal is arranged between the actuation element and the valve housing that seals a gap between the actuation element and the valve housing in both positions of the switch shaft. The actuation element is preferably designed in such a way that the switch shaft can be brought from the first position into the second position or from the second position into the first position by actuating the actuation element. The actuation element is preferably actuated by applying a force. The actuation element is then a push button or another push element, wherein the applied force preferably acts along the longitudinal direction of the switch shaft,

The actuation element is preferably connected to the switch shaft in such a way that the actuation element is also moved when the switch shaft is brought out of the first position into the second position or from the second position into the first position. The actuation element is preferably at least partially arranged in a depression of the valve housing. Particularly preferably, the actuation element is completely arranged in the depression of the valve housing in at least one of the two positions of the switch shaft. This means that it does not protrude beyond the outer contour of the valve housing. There is a gap between the valve housing and the switch shaft that is sealed against the environment by a seal, preferably a ring seal.

Preferably, the switch shaft has a first sealing element, preferably a first sealing ring, which fluidically separates the inlet connection from the outlet connection when the switch shaft is in the second position. Such an embodiment with a single ring seal is known from the prior art. If the switch shaft is moved, the first sealing element also moves and thus frees the flow path from the inlet connection to the outlet connection. The shaft chamber, in which the switch shaft is located and through which the hydraulic fluid flows when the switch shaft is in the first position, is sealed by two boundary seals in such a way that hydraulic fluid cannot leave this chamber. The seal that seals the gap between the actuation element and the valve housing is provided in addition to the first sealing element and the boundary seals.

In one preferred embodiment, an actuation element is arranged at each end of the switch shaft, wherein a seal is arranged between each actuation element and the valve housing that seals a gap between the respective actuation element and the valve housing in both positions of the switch shaft. Particularly preferably, the two actuation elements are push buttons which are configured in such a way that a pressure applied in the axial direction in terms of the longitudinal direction of the switch shaft causes the switch shaft to be brought from the first position into the second position or from the second position into the first position. Particularly preferably, each actuation element is to be used for only one direction of this change in position. For example, the actuation element is configured at a first end of the switch shaft such that actuating the actuation element moves the switch shaft out of the first position into the second position. The actuation element at the opposite second end of the switch shaft is then preferably configured such that actuating this actuation element moves the switch shaft out of the second position into the first position.

There is preferably a volume enclosed between the valve housing and each of the actuation elements so that there are preferably two volumes, each of which is located at one end of the switch valve and is enclosed between the valve housing and one of the two actuation elements. The volume is sealed and enclosed by the respective seal, which seals the gap between the respective actuation element and the valve housing. In one preferred embodiment, the two volumes are fluidically connected to each other by a channel, which is preferably provided in the switch shaft or in the valve housing and preferably extends across the entire length of the switch shaft.

The switch shaft can preferably be brought from the first position into the second position or from the second position into the first position by displacing it along its longitudinal direction. Particularly when the displacement of the switch shaft also means the displacement of the actuation elements, this causes one of the two volumes to decrease in size and the other to increase in size. Since both volumes are sealed against the surroundings, this would result in a change in pressure, which would be caused by a force that acts against the movement of the switch shaft that causes this change in the two volumes. This effect can be avoided by way of the channel provided in the switch shaft that connects the two volumes to one another.

If an actuation element is actuated by applying a pressure and the switch shaft displaced along its longitudinal axis, the volume between the actuated actuation element and the valve housing is reduced. Since the other actuation element, which is arranged at the opposite end of the switch shaft, moves at the same time, the volume between the other actuation element and the valve housing increases. Preferably, the volumes are designed in such a way that the overall volume-made up of the two volumes and the volume of the channel-remains constant irrespective of the position of the switch shaft. In this way, the pressure within this overall volume can also be kept constant and a force caused by pressure differences cannot occur.

In one advantageous embodiment, the switch shaft can be brought from the first position into the second position or vice-versa by rotating it about its longitudinal axis. In this case the first seal, which separates the inlet connection from the outlet connection when the switch shaft is in the second position, is preferably a sloping ring seal, such as an O ring. The switch shaft preferably has a circumferential groove in which the ring seal is arranged. While in the prior art such a groove forms a circle in a plane that is perpendicular to the longitudinal extension of the switch shaft, in the embodiment described here said plane is preferably tilted, particularly preferably at an angle of 45°. If the switch shaft is rotated in this position, for example by 180° about its longitudinal direction, its position and orientation relative to the valve housing changes. The position at which the inlet connection and the outlet connection open into the shaft chamber, also known as the point of entry, is chosen so that they are located on different sides of the first seal, preferably the O-ring, in the second position of the switch shaft and thus also of the first seal, and on the same side of the first seal in the first position of the switch shaft.

Preferably, the first sealing element comprises a seal arranged on the switch shaft in such a way that the plane defined by the sealing ring forms an angle of at least 20°, preferably at least 30°, especially preferably at least 40°, and at most 70°, preferably at most 60°, especially preferably 50°. The sealing ring preferably lies entirely in a plane that is thus defined by said sealing ring. Sealing rings are usually arranged in such a way that the switch shaft extends orthogonally to the plane defined by the sealing ring. This is different in this embodiment example. Due to the rotation of the switch shaft in the shaft chamber relative to the valve housing, the point on the wall of the shaft chamber that comes into contact with the sealing ring changes. In the first position of the switch shaft, this contact point extends along a first line. In the second position of the switch shaft, which is achieved in this embodiment example by rotating the switch shaft about its longitudinal axis, this contact point extends along a second line, which is different from the first line. The first line and the second line lie on different sides of the inlet connection or the outlet connection.

In this case, the actuation element is preferably a rotary knob which, for example can be actuated manually by rotating it. Preferably, the rotary knob is connected to the switch shaft in a torque-proof manner such that a rotation of the actuation element with this design also results in a rotation of the switch shaft, which is therefore brought from the first position into the second position or vice-versa. As an alternative, the actuation element is designed as a push button that is actuated by applying a force that acts in a longitudinal direction of the switch shaft. Preferably, the switch shaft has a thread at the end where such an actuation element is arranged. The actuation element has a corresponding counter-thread. The thread is preferably configured as an outer thread on the switch shaft. Correspondingly, the counter-thread is preferably an inner thread. The thread and counter-thread are configured in such a way that no self-locking effect occurs. A movement of the actuation element in the axial direction of the switch shaft caused, for example, by applied pressure, is then translated by the two threads into a rotational movement of the switch shaft, causing it to be brought from the first position into the second position or vice-versa.

In one particularly preferable embodiment, the switch shaft comprises an actuation element at each end, said actuation elements being designed as such a push element, for example a push button. The switch shaft then preferably has a thread at both ends and both actuation elements have a corresponding counter-thread. If one of the actuation elements is now displaced in the axial direction, preferably pushed into the valve housing, this longitudinal movement is translated into a rotational movement of the switch shaft. This preferably causes the other actuation element arranged at the opposite end to also move longitudinally and, particularly preferably, to move out of the depression of the valve housing. As a result, with this embodiment it is also possible to ensure that there is a volume between one of the actuation elements and the valve housing, as described above, whereby one of these volumes is reduced and the other volume is increased when the switch shaft is moved from the first position to the second position or vice versa.

Alternatively, the two actuation elements are arranged in such a way that they move towards each other when the pressure is applied to one of the actuation elements. This means that both actuation elements move relative to another element of the switch shaft, for example the switch shaft or the valve housing. This can be achieved, for example, by providing the switch shaft with a thread at both ends that run in opposite directions to each other. Each actuation element also has a thread, wherein these threads also run in opposite directions to each other.

Advantageously, at least one permanent magnet is arranged on at least one of the actuation elements and/or on the valve housing, said magnet generating a force with a corresponding counter-element that pre-tensions the switch shaft in the first direction or the second direction. For example, the counter-element is designed as a magnetic element; for example, it is made from a magnetizable metal. If the permanent magnet is moved close to the counter-element, an attractive force is generated as a result of the magnetic interaction. Alternatively or additionally, the counter-element is likewise designed as a permanent magnet. In this case, it is arranged such that unlike poles are directed towards each other, thus generating an attractive interaction between the two permanent magnets. Preferably, multiple such pairs of permanent magnet and counter-element are provided.

Particularly where one or both of the actuation elements is/are moved into a recess in the valve housing or otherwise moved towards the valve housing upon actuation, it makes sense to generate a magnetic interaction and to arrange corresponding components, as described above. In this case, the magnetic attractive interaction is always at its greatest when the respective actuation element has been actuated and the switch shaft has reached its new position. As a result, the interaction and the resulting force hold the switch shaft in its new position, thereby making it more difficult or completely impossible for the switch shaft to be inadvertently removed from this position.

Preferably, the point of entry of the inlet connection into the shaft chamber and/or the point of entry of the outlet connection into the shaft chamber is rounded off with a circumferential bore. This means that the diameter of the respective connection increases in the area of the point of entry. In addition, at the transition point between the wall of the shaft chamber and the wall of the respective connection there is no sharp edge, but a rounding. If the switch shaft is brought from the first position into the second position or vice-versa, the first sealing element has to be moved with the switch shaft. In the process, it is moved away via the inlet connection or the outlet connection. The rounding of the entry point means that the first sealing element is subject to significantly less mechanical stress during this movement and therefore protected. This rounding, which can also be referred to as a depression, is preferably a spherical depression. The sealing ring preferably used as a first sealing element, especially an O ring, then slides more effectively over these entry points and the seal exhibits less wear. It also means easier movement and a lower risk of jamming when moving the switch shaft from one position to the other.

This embodiment is advantageous, irrespective of whether a gap between the actuation element and the valve housing is sealed by a seal or the design of such a seal.

A prosthetic foot with a foot part, a lower leg part pivotably arranged on the foot part, and a hydraulic system, the hydraulic system comprising two hydraulic chambers that are fluidically connected by a hydraulic line, wherein a switch valve is located in the hydraulic line that comprises a valve housing with an inlet connection and an outlet connection and a switch shaft that can be moved in a shaft chamber of the valve housing, wherein said switch shaft can be brought into a first position, in which the inlet connection is fluidically connected to the outlet connection, and into a second position, in which the inlet connection is fluidically separated from the outlet connection, is therefore a separate invention if the point of entry of the inlet connection and/or the outlet connection comprises a groove, rounding or depression as described here.

The features described in this application can be combined with this separate invention individually, in any conceivable combination or completely.

Preferably, the actuation element is actuated by displacing it, wherein it is preferably displaced parallel to the longitudinal direction of the switch shaft. In this case, it is advantageous if the respective actuation element is guided in a mount on the valve housing such that it cannot twist. As a result, the actuation element cannot be rotated or can only be rotated in a predetermined shape in addition to the longitudinal displacement that is required to actuate the actuation element. This can be achieved particularly easily by ensuring that the actuation element and the mount in which it can be moved comprise a correspondingly designed cross-section, which is not circular. Preferably, it is oval and/or has at least one guide projection, such as a guide pin. This projection or pin is arranged in a corresponding depression or groove in the wall of the mount and prevents the actuation element from twisting relative to the mount.

Preferably, the switch valve, particularly preferably the switch shaft, has a resetter that is configured to move the switch shaft from the first position into the second position when the switch shaft has been in the first position for a set amount of time. In the second position, a through-flow of hydraulic fluid through the switch valve is not possible or is only possible to a limited extent. In the majority of orthopedic devices, this means that a movement between two connected components is not possible. This increases stability and is therefore usually referred to as the default state. It is advantageous if this default state can be reached and assumed automatically. As a result, the switch shaft is moved from the first position, in which hydraulic fluid can pass through the valve, into the second position once the switch shaft has been in the first position for the set amount of time. This is also referred to as a time delay or time delay element.

It preferably refers to a hydraulic time delay which, particularly preferably, is adjustable. In this case, adjustability refers to the length of time once the switch shaft has been brought into the second position. Alternatively or additionally, the resetter has a mechatronic drive that moves the switch shaft into the first and/or the second position.

In one preferred embodiment, the switch shaft comprises a section with a reduced cross-section, such as a flattening, which is formed on one or both sides, for example, or a recess, groove and/or a symmetrical or asymmetrical bore relative to the central axis of the switch shaft, through which hydraulic fluid can flow from the inlet connection to the outlet connection when the switch shaft is in the first position. Preferably, the switch shaft is spring-loaded in the first position and/or the second position. A spring used for this purpose preferably provides support for switching the switch shaft into the locking second position and/or holding the first and/or the second position. The spring is preferably arranged in a volume between an actuation element and the valve housing. A spring element is preferably arranged between the valve housing and each actuation element provided.

In the following, a number of embodiment examples of the invention will be explained in more detail with the aid of the accompanying figures. They show:

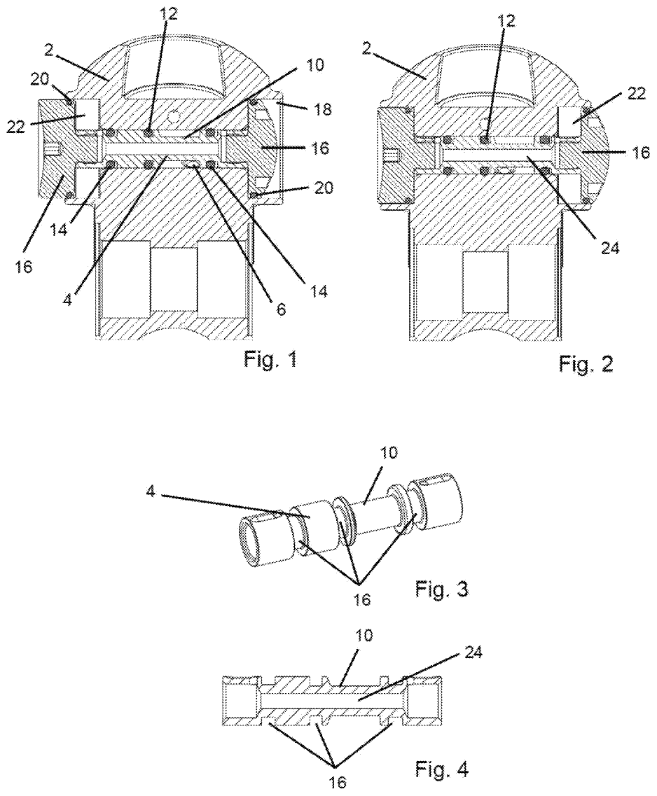

FIGS. 1 and 2—a switch valve according to a first embodiment example of the present invention with the switch shaft in various positions,

FIGS. 3 and 4—the switch shaft from FIGS. 1 and 2 in a 3-dimensional and a sectional view,

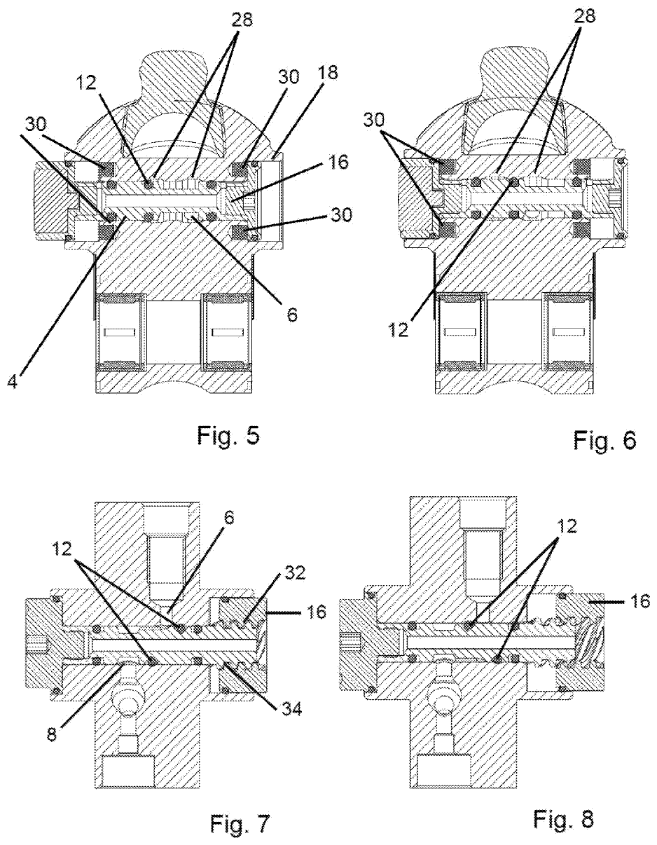

FIGS. 5 and 6—a switch valve according to a further embodiment example with the switch shaft in various positions,

FIGS. 7 and 8—a switch valve according to a further embodiment example with the switch shaft in various positions,

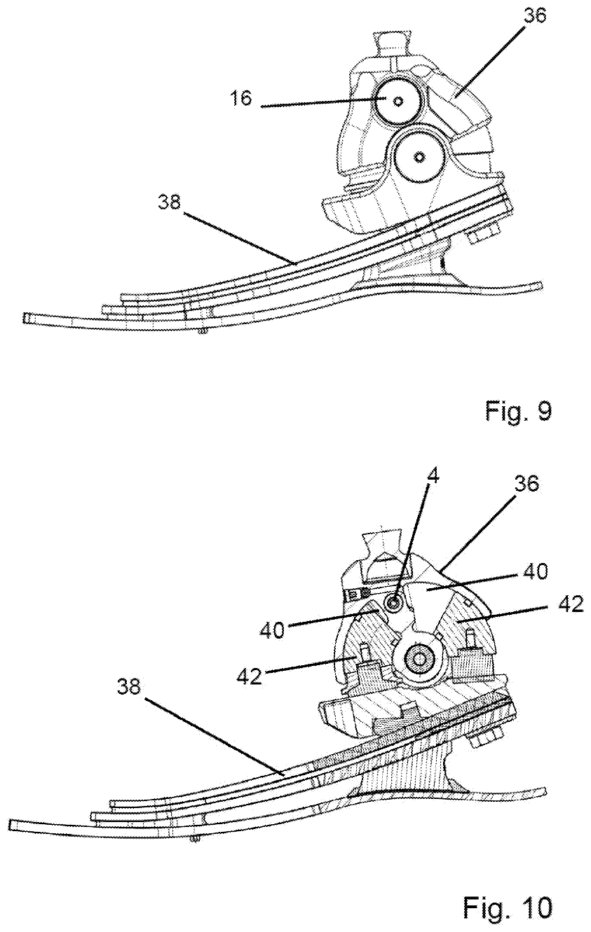

FIGS. 9 and 10—a prosthetic foot containing a switch valve in a side view and a sectional view,

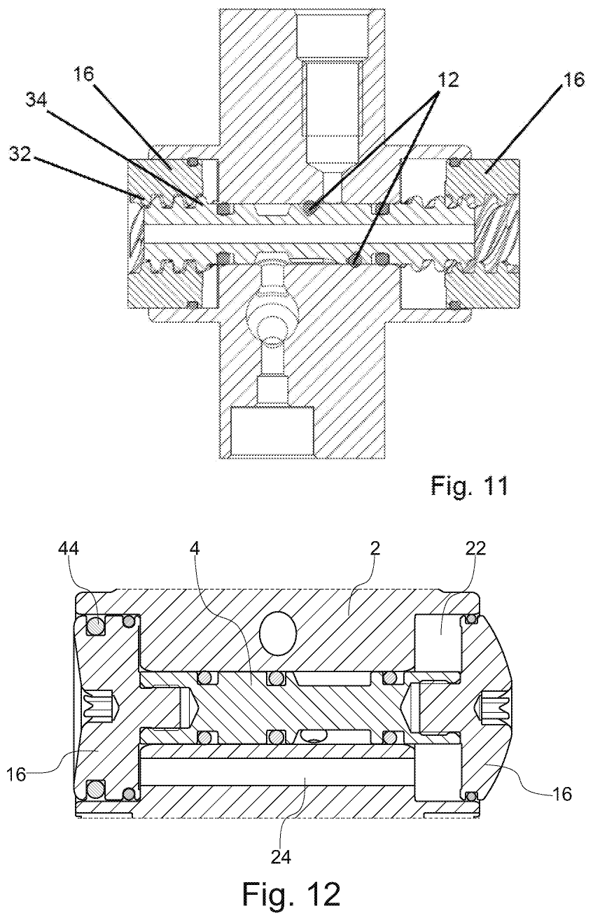

FIG. 11—the schematic view through a switch valve according to a further embodiment example of the present invention and

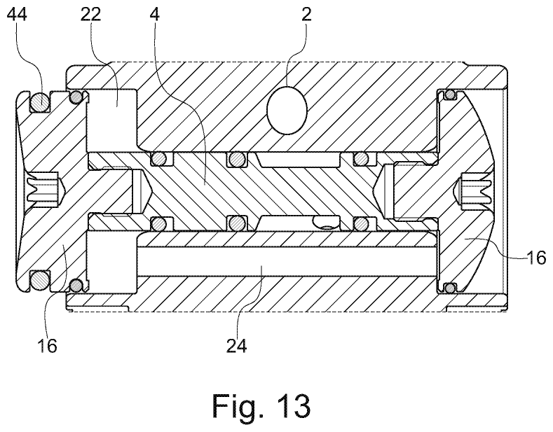

FIGS. 12 and 13—a section from a sectional view through a switch valve according to a further embodiment example of the present invention.

FIG. 1 depicts a switch valve according to a first embodiment example of the present invention. It comprises a valve housing 2 in which a switch shaft 4 is located. In the embodiment example shown, the switch shaft 4 can be displaced left and right in a shaft chamber. FIG. 1 shows the switch shaft in the first position. An inlet connection 6 opens into the shaft chamber. An outlet connection 8, not depicted in FIG. 1, also opens into the shaft chamber; the associated entry point is not visible in the view shown in FIGS. 1 and 2. In FIGS. 1 and 2, the switch shaft 4 has a tapering 10, i.e. a section with a reduced cross-section. In the situation depicted in FIG. 1, in which the switch shaft 4 is in the first position, hydraulic fluid entering from the inlet connection 6 into the shaft chamber can flow to the outlet connection in the area of the tapering 10 between the switch shaft 4 and the wall of the shaft chamber.

The switch shaft comprises a first sealing element 12 in the form of a sealing ring surrounding the switch shaft 4, preferably an O ring. The entry point of both the inlet connection 6 and the outlet connection 8 is located on the same side of the first sealing element 12; in the embodiment example shown, they are to the left. A flow of fluid from the inlet connection 6 to the outlet connection 8 is therefore not prevented by the first sealing element 12 in the situation in FIG. 1. The shaft chamber, which may contain hydraulic fluid, is restricted and sealed on both sides by a boundary seal 14.

An actuation element 16 is arranged at each end of the switch shaft 4, the former being guided in a recess 18 of the valve housing 2 provided for this purpose. In FIG. 1, one can see that the switch shaft has been displaced to the left into the first position so that the left-hand actuation element 16 protrudes from its recess 18. The right-hand actuation element 16, on the other hand, has been moved into its recess 18. The gap between the actuation element 16 and the valve housing 2 is sealed respectively by a seal 20.

If in the situation shown in FIG. 1 a pressure directed along the longitudinal direction of the switch shaft 4—to the right in the embodiment example shown—is applied to the left-hand actuation element 16, the switch shaft 4 is brought from the first position depicted in the figure into the second position, which is illustrated in FIG. 2.

It is clear that in FIG. 2 the left-hand actuation element 16 has been moved into its recess 18 and that the right-hand actuation element 16 protrudes beyond the rim of its recess 18. The displacement of the switch shaft 4 has also caused the first sealing element 12 to be displaced. It is now between the entry point of the inlet connection 6 and the entry point of the outlet connection 8, thereby blocking a flow of hydraulic fluid through the shaft chamber.

In FIG. 1 there is a volume 22 between the left-hand actuation element 16 and the valve housing 2. This volume 22 is reduced when the switch shaft 4 is displaced into the second position. FIG. 2 shows that there is a volume 22 between the right-hand actuation element 16 and the valve housing 2 when the switch shaft 4 is in the second position. By carefully selecting the dimensions of the actuation elements 16 and the recesses 18, the combined overall size of the two volumes 22 is constant. In order to enable a balance of a fluid, such as air, between the two volumes 22, the switch shaft 4 has a channel 24 that connects the two volumes 22 to each other.

FIG. 3 shows the switch shaft 4 in a 3-dimensional view. The tapering 10 can be seen. Three grooves 26 can also be seen. The first sealing element 12 is arranged in the central groove 26 and a boundary seal 14 is arranged in each of the other grooves 26. FIG. 4 depicts the switch shaft 4, in which the channel 24 can also be seen, in a sectional view.

FIGS. 5 to 6 show a further embodiment example of the present invention. In terms of its function, it corresponds to the switch valve from FIGS. 1 and 2. Therefore only differences will be described.

The switch shaft 4 is arranged in a shaft chamber that has two indentations 28. In FIG. 5, the switch shaft 4 is depicted in the first position. The entry point of the inlet connection 6 and the entry point of the outlet connection, not depicted here, are located on the same side of the first sealing element 12 (to the right in the embodiment example shown). It is in the area of one of the two indentations 28 and, in the embodiment example shown, it does not have any contact with the wall of the shaft chamber. It is therefore possible for hydraulic fluid to flow from the inlet connection to the outlet connection. In FIG. 5, the right-hand actuation element 16 is arranged in its mount 18. Two magnets 30 are positioned at the base of each of the two recesses 18. The actuation elements 16 are made of a magnetic or magnetizable material or contain such a material, so that a magnetic attraction between the magnets 30 and the actuation elements stabilizes the respective position of the switch shaft 4.

In FIG. 6, the switch shaft 4 is depicted in its second position. The first sealing element 12 is now arranged between the entry point of the inlet connection 6 and the entry point of the outlet connection 8, thereby blocking a flow of fluid through the shaft chamber. In this position of the switch shaft 4, the first sealing element 12 comes into contact with the wall of the shaft chamber between the two indentations 28. In FIG. 6, the left-hand magnets 30 exert a force of attraction on the left-hand actuation element 16, holding it and therefore the switch shaft 4 in the second position shown.

FIGS. 7 to 8 show a switch valve according to a further embodiment example of the present invention. In FIG. 7 the switch shaft 4 is shown in the first position and in FIG. 8 it is shown in the second position. As in the previous embodiment examples, the first sealing element 12 is designed as an O ring; however, it is arranged at a slope or tilt on the switch shaft. In FIG. 7, the entry point of the inlet connection 6 and the entry point of the outlet connection 8 are located on the same side of the first sealing element 12 (to the left in the embodiment example shown) such that it cannot prevent a flow of fluid through the switch valve. The actuation element 16 is fitted with an inner thread 32. The switch shaft 4 has a corresponding outer thread 34. The threads 32, 34 are not designed to be self-locking.

In FIG. 8, the switch shaft 4 is depicted in its second position. The first sealing element 12 has been rotated with the switch shaft 4 and now extends between the entry point of the inlet connection 6 and the entry point of the outlet connection 8, thereby blocking the flow of fluid through the valve. If, in the second position of the switch shaft 4 shown in FIG. 8, a pressure is applied to the actuation element 16, the actuation element 16 in the embodiment example shown is displaced to the left. Due to the engaged threads 32, 34, this displacement of the actuation element 16 leads to a rotation of the switch shaft 4 about its longitudinal axis, causing it to move from the second position depicted into the first position.

FIG. 9 depicts a prosthetic foot with a lower leg part 36 and a foot part 38. Both are pivotably arranged on each other, wherein hydraulic fluid has to be moved through a switch valve according to one embodiment example of the present invention, said valve being installed in the prosthetic foot, when the two components are swivelled relative to one another. One of the actuation elements 16 can be seen.

FIG. 10 depicts the prosthetic foot in a sectional view. The lower leg part 36 and the foot part 38 can be seen. In addition, the two hydraulic chambers 40 are shown which, in the embodiment example shown, are restricted by boundary elements 42. One can recognize the switch shaft 4 in the wall between the two hydraulic chambers 40, said wall acting as a piston, the former extending perpendicular to the drawing plane in the diagram.

FIG. 11 is similar to the diagram in FIG. 8, wherein in FIG. 11 an actuation element 16 is arranged at each end of the switch shaft. Both ends of the switch shaft 4 have an outer thread 34 arranged on the outside of the switch shaft 4 and the two actuation elements comprise a correspondingly designed inner thread 32 arranged on the inside. If the two outer threads 34 are designed to run in the opposite direction to one another, a pressure on one of the two actuation elements 16 causes both actuation elements 16 to be displaced inwards, i.e. towards each other. The actuation element 16 to which pressure is not applied is otherwise moved outwards. In this preferred embodiment, the air can flow out of the right-hand chamber 22 into the left-hand chamber 22 and vice-versa. A connection to the atmosphere is therefore not necessary.

FIG. 12 depicts a section of a sectional view through a switch valve according to a further embodiment example of the present invention. Unlike in the other embodiment examples shown, the channel 24 is not in the switch shaft 4, but in the valve housing 2. It connects the two volumes 22, of which only the volume 22 shown on the right is designated in the position of the switch shaft 4 shown. One can also see a signal ring 44 on the left-hand actuation element 16, said ring being designed as a color-contrasting element, for example red or yellow. It can be an O ring, for example made of an elastic material. In the position of the switch shaft 4 shown in FIG. 12, the signal ring 44 cannot be seen from the outside. If the right-hand actuation element 16 is actuated and the switch shaft 4 in FIG. 12 displaced to the left, the left-hand actuation element 16 is also displaced to the left and the signal ring 44 becomes visible from the outside. This situation is shown in FIG. 13. The switch shaft 4 is in the first position, in which the foot part 38 and the lower leg part 36, not depicted in FIG. 12, can be moved relative to each other. In this state, it may be dangerous to subject the prosthetic foot to a load, for example in that the wearer of the foot treads, stands up or walks. Due to the signal ring 44, the wearer receives a visible signal that the switch shaft 4 first has to be displaced into its second position, which is shown in FIG. 12.

### REFERENCE LIST

2 valve housing
4 switch shaft
6 inlet connection
8 outlet connection
10 tapering
12 first sealing element
14 boundary seal
16 actuation element
18 recess
20 seal
22 volume
24 channel
26 groove
28 indentation
30 magnet
32 inner thread
34 outer thread
36 lower leg part
38 foot part
40 hydraulic chamber
42 boundary element
44 signal ring

1. A prosthetic foot, comprising: with
a foot part,
a lower leg part pivotably arranged on the foot part,
a hydraulic system comprising two hydraulic chambers that are fluidically connected by a hydraulic line,
a switch valve is located in the hydraulic line, wherein the switch valve comprises
a valve housing with an inlet connection and an outlet connection, and
a switch shaft that is movable in a shaft chamber of the valve housing, wherein the switch shaft
can be brought into a first position in which the inlet connection is fluidically connected to the outlet connection, and
can be brought into a second position, in which the inlet connection is fluidically separated from the outlet connection, and
at least one actuation element arranged at at least one end of the switch shaft wherein at least one a seal is arranged between the actuation element and the valve housing, wherein the seal seals a gap between the actuation element and the valve housing in both the first position and the second position of the switch shaft

2. The prosthetic foot according to claim 1, wherein the at least one actuation element includes a first actuation element and a second actuation element respectively is arranged at each end of the switch shaft, wherein the at least one seal comprises a seal is arranged between each actuation element at each end of the switch shaft and the valve housing, wherein the at least one seal seals a gap between the actuation element and the valve housing at each end of the switch shaft.

3. The prosthetic foot according to claim 2, wherein a volume is enclosed between the valve housing and each of the first actuation element and the second actuation element and forms two volumes, wherein the two volumes are fluidically connected to each other by a channel.

4. The prosthetic foot according to claim 1, wherein the switch shaft is moveable from the first position into the second position by displacing the switch shaft along a longitudinal direction of the switch shaft.

5. The prosthetic foot according to claim 1, wherein the switch shaft is moveable can be moved from the first position into the second position by rotating the switch shaft it about a longitudinal direction of the switch shaft.

6. The prosthetic foot according to claim 5, wherein the at least one seal comprises at least one seal ring arranged on the switch shaft such that a plane defined by the at least one sealing ring forms an angle of at least 20°, and at most 70°.

7. The prosthetic foot according to claim 1, wherein the switch shaft is rotatable about a longitudinal axis by applying a pressure along a longitudinal direction of the switch shaft to the at least one actuation element.

8. The prosthetic foot according to claim 7, wherein the at least one actuation element includes two actuation elements, and wherein both actuation elements move towards each other when the pressure is applied to one of the two actuation elements.

9. The prosthetic foot according to claim 1, further comprising least one permanent magnet is arranged on at least one of
the at least one actuation element elements and/or
the valve housing,
wherein said at least one permanent magnet generating a force with a corresponding counter-element that pre-tensions the switch shaft in a first direction or in a second direction.

10. The prosthetic foot according to claim 1, wherein a point of entry of the inlet connection into the shaft chamber and/or a point of entry of the outlet connection into the shaft chamber are rounded with a circumferential bore.

11. The prosthetic foot according to claim 1, wherein the at least one actuation element is guided in a mount of the valve housing such that the at least one actuation element cannot twist.

12. The prosthetic foot according to claim 1, wherein the the switch valve, has a resetter that is configured to set the switch shaft back from the first position into the second position once the switch shaft has been in the first position for a set amount of time.

13. The prosthetic foot according to claim 12, wherein the resetter comprises a hydraulic time delay.

14. The prosthetic foot according to claim 12, wherein the resetter comprises a mechatronic drive.

15. The prosthetic foot according to claim 1, wherein the switch shaft comprises a section with a reduced cross-section, through which hydraulic fluid can flow from the inlet connection to the outlet connection when the switch shaft is in the first position.

16. The prosthetic foot according to claim 3 the channel (24) is in the switch shaft or in the valve housing.

17. The prosthetic foot according to claim 11 wherein the mount has an oval outer contour and/or a guide projection.

18. The prosthetic foot according to claim 17 wherein the guide projection is a guide pin.

19. The prosthetic foot according to claim 13 wherein the hydraulic time delay is adjustable.

20. The prosthetic foot according to claim 15 wherein the reduced cross-section is selected from the group consisting of a recess, groove, a symmetrical or asymmetrical bore relative to the central axis of the switch shaft, and a flattening.
